(19) United States
(12) Patent Application Publication
(10) Pub. No.: US 2013/0053598 A1
(43) Pub. Date: Feb. 28, 2013

(54) METHOD FOR PRODUCING A CROSS-COUPLING PRODUCT OF A BENZENOID DIAZONIUM SALT

(71) Applicants: Nicolas Boege, Hamburg (DE); Andreas Kreipl, Hamburg (DE); Bernd Schmidt, Potsdam (DE); Frank Hoelter, Potsdam (DE); René Berger, Berlin (DE)

(72) Inventors: Nicolas Boege, Hamburg (DE); Andreas Kreipl, Hamburg (DE); Bernd Schmidt, Potsdam (DE); Frank Hoelter, Potsdam (DE); René Berger, Berlin (DE)

(73) Assignee: ZYLUM BETEILIGUNGSGESELLSCHAFT MBH & CO. PATENTE II KG, Schonefeld/Waltersdorf (DE)

(21) Appl. No.: 13/265,736

(22) PCT Filed: Apr. 22, 2010
(86) PCT No.: PCT/EP2010/055347

§ 371 Date: Nov. 12, 2012

(30) Foreign Application Priority Data
Apr. 22, 2009 (EP) 09158489.6

(51) Int. Cl. C07C 67/293; C07C 37/11; C07C 41/48; C07C 67/343; C07C 201/12

(52) U.S. Cl. 560/23; 560/55; 560/75; 568/585; 560/255; 568/592; 568/707; 568/767; 568/747; 560/67

(57) ABSTRACT

The invention relates to a method for producing a cross-coupling product of a benzenoid dizonium salt according to the general formula (I), wherein the groups R₁, R₂, R₃, R₄, and R₅ represent hydrogen, halogen, an alkyl, alkenyl, aryl, alkoxy, aryloxy, nitro, cyano, hydroxy, acetyl, and/or diazo groups independently of each, and X represents BF₄, Cl, F, SO₃CH₃, CO₂CH₃, PF₆, ClO₂CH₃, or CIO₄, comprising the following steps: (a) providing a benzenoid amide, which with the exception of the diazo function has the same substituents R₁, R₂, R₃, R₄, and R₅ as the benzenoid diazonium salt of the general formula (I), and hydrolytically cleaving the amide to form an amine or providing a corresponding amine, (b) diazotizing the amine thus obtained or provided with a nitrite, and (c) subsequently reacting the benzenoid diazonium salt with a coupling partner in the presence of a catalyst to form a cross-coupling product, wherein the coupling parter is represented by the general formula (II), R₆, R₇, and R₈ are the same or different and represent hydrogen, carboxyalkyl groups, carboxyaryl groups, alkyl groups, aryl groups, alkoxy groups, aryloxy groups, wherein the groups can each contain Si, N, S, O, and or halogen atoms, or R₆ and R₇ with the double bound form an aromatic ring, which can be provided with R₈ and one to four further substituents, independently of each other, selected from the group comprising a straight-chain or branched (C₁-C₆) alkyl group, a (C₃-C₇) cycloalkyl group, a straight-chain or branched (C₁-C₆) alkenyl group, a straight-chain or branched (C₁-C₆) alkyoxy group, halogen, the hydroxy group, an amino, di(C₁-C₆) alkylamino, nitro, acetyl, cyan, benzyl, 4-methoxybenzyl, 4-nitrobenzyl, phenyl, and 4-methoxyphenyl group and represents Y=H, —B(OR)₂, —SnR₃, —ZnR, —SiR₃, or Mg (halogen), and wherein at least the steps (b) and (c) are performed without intermediate isolation of an intermediate product. According to said method, cross-couplings can be performed more simply and with improved yield without the hydroxyl group in aromatic reactants containing hydroxyl groups having to be provided with a protective group.

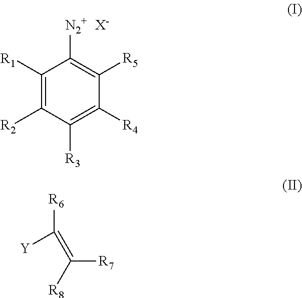

The invention relates to a method for producing a cross-coupling product of a benzenoid diazonium salt, wherein a benzenoid amide is provided and is hydrolytically cleaved to an amine or the corresponding amine is already present as such, the amine is diazotized with a nitrite and the diazonium salt obtained is reacted with a coupling partner, in the presence of a catalyst, to a cross-coupling product.

Benzenoid diazonium salts are known in the prior art. Thus, p-benzyloxyphenyldiazonium tetrafluoroborate is disclosed for example in DE 10 2006 053 064 A1. This compound has a protective group for the hydroxyl group of the skeleton structure. The protective groups should be selected so that on the one hand they resist the hydrolytic cleavage of the amide group and on the other hand they oppose the diazotization reaction. Such protective groups are known per se by a person skilled in the art and are described for example in “Protective Groups in Organic Chemistry” by Theodora W. Greene, Wiley Publishers, 1981.

The aforementioned fluoroborate compound is a reactive starting compound, which can be reacted in a manner known per se. DE 10 2006 053 064 A1 refers for example to the Japp-Klingemann reaction, deamination, Sandmeyer reaction, Schiemann reaction, Meerwein reaction and Gomberg-Bachmann reaction, it being said that the diazonium salts mentioned in this prior art are suitable especially for the Heck reaction. The known teaching described above is disadvantageous for the following reasons: before conversion to a diazonium salt the phenolic starting compound must be provided with a protective group. The salt is then used in situ in cross-coupling reactions, and then the protective group is removed. Clearly this method is expensive. A similar situation follows from EP 1 253 466 B1.

Methods of production for phenol-containing target compounds, for which the use of protective groups can be avoided, are also known from the prior art.

Thus, the reference “Tetrahedron Lett.”, 1979, 657-660 describes a method of synthesis of biaryls, which takes place mechanistically as a Gomberg-Bachmann reaction or in the intramolecular variant as a Pschorr cyclization. This starts from a phenyldiazonium tetrafluoroborate, which is reduced in the presence of stoichiometric amounts of a titanium(III) compound with splitting-off of nitrogen, and the resultant aryl radical reacts upon a phenol, preferably in the ortho position. However, the regioselectivity of the reaction described is not perfect. Then oxidation of the new radical takes place, with splitting-off of a proton and formation of the biaryl. Important disadvantages of this method are on the one hand the use of an equivalent of an air-sensitive and expensive titanium(III) compound, which leads subsequently to the formation of equimolar amounts of metallic waste products (titanium(IV) compounds), requiring expensive separation. On the other hand the reaction takes place in the presence of an acid, so that there is limited tolerance to functional groups. Finally the yield is at most only 31%.

Another relevant piece of prior art follows from US 2003/0120124 A1. This describes the use of diazonium salts as substrate in coupling reactions. For example, a coupling reaction is proposed, in which phenyltrimethylsilane is reacted with aryldiazonium salts, corresponding to the type of a Hiyama. coupling. This prior art is not faced with the problem of using phenolic compounds in the coupling reaction and providing their hydroxyl group with a protective group, as proposed in the prior art, and only then allowing the desired coupling reactions to take place. Similarly, the use of 2-nitro-substituted phenyldiazonium salts as substrate in Suzuki cross couplings was described in “Advanced Synthesis & Catalysis”, 2008, 350 (10), 1577-86.

Starting from the prior art presented above, the problem to be addressed by the invention is to offer a technical proposal, according to which cross couplings can be applied more easily and with improved yield, without needing to provide the hydroxyl group in hydroxyl group-containing aromatic, educts with a protective group. Moreover, the invention should offer advantages over the prior art, in which the hydroxyl groups are not contained in the aromatic diazonium salt, but in the coupling partner, which, however, display poor regioselectivity within the coupling reaction.

According to the invention, this problem is addressed by a method for producing a cross-coupling product using a benzenoid diazonium salt according to general formula (I)

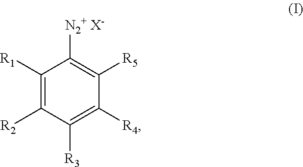

where the groups R₁, R₂, R₃, R₄ and R₅, independently of one another, represent hydrogen, halogen, an alkyl, alkenyl, aryl, alkoxy, aryloxy, nitro, cyano, hydroxy, acetyl and/or diazo group, and X represents BF₄, Cl, F, SO₃CH₃, CO₂CH₃, PF₆, ClO₂CH₃ or ClO₄, comprising the steps (a) providing a benzenoid amide of formula (I), which with the exception of the diazo function has the same substituents R₁, R₂, R₃, R₄ and R₅ as the benzenoid diazonium salt of general formula (I), and hydrolytic cleavage of the amide to an amine or providing a corresponding amine, (b) diazotizing the amine, thus obtained or provided, with a nitrite and (c) then reacting the benzenoid diazonium salt with a coupling partner in the presence of a catalyst with formation of a cross-coupling product, the coupling partner being represented by general formula (II)

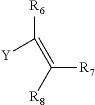

and R₆, R₇ and R₈ are identical or different and represent hydrogen, carboxyalkyl groups, carboxyaryl groups, alkyl groups, aryl groups, alkoxy groups, aryloxy groups, wherein the groups can in each case contain Si, N, S, O and/or halogen atoms, or R₆ and R₇ form, with the double bond, an aromatic ring, which can be provided with R₈ and one to four further substituents, independently of one another, selected from the group comprising a linear or branched (C₁-C₆)-alkyl group, a (C₃-C₇)-cycloalkyl group, a linear or branched (C₁-C₆)-alkenyl group, a linear or branched (C₁-C₆)-alkoxy group, halogen, the hydroxyl group, an amino, di(C₁-C₆)-alkylamino, nitro, acetyl, cyano, benzyl, 4-methoxybenzyl, 4-nitrobenzyl, phenyl and 4-methoxyphenyl group, and Y=H, —B(OR)₂, —SnR₃, —ZnR, —SiR₃ or Mg (halogen) and wherein at least the steps (b) and (c) are carried out without interposed isolation of an intermediate.

The above presentation comprises various concrete groups. These will not be described in more detail. On the other hand, however, it also comprises general concepts, which can advantageously be described concretely in the context of the invention. Thus, it has proved to be desirable if the alkyl group represents a linear or branched (C₁-C₆)-alkyl group, a (C₃-C₇)-cycloalkyl group, the alkenyl group represents a linear or branched (C₁-C₆)-alkenyl group, the alkoxy group represents a linear or branched (C₁-C₆)-alkyloxy group, the aryl group represents a benzyl, 4-methoxybenzyl, 4-nitrobenzyl, phenyl or 4-methoxyphenyl group and if halogen denotes fluorine, chlorine or bromine.

It is preferable if, in the benzenoid diazonium salt of formula (I), at least one of the groups R¹, R², R³, R⁴ or R⁵ has an oxygen atom, which is joined to the aromatic ring in formula (I). It is especially preferable if the benzenoid diazonium salt is a phenolic diazonium salt, in which at least one of the groups R¹, R², R³, R⁴ or R⁵ represents a hydroxyl group. It is desirable for the phenolic diazonium salt not to have a protective group. Especially preferably, R³ is a hydroxyl group. In some cases it is also advantageous if R¹, R², R⁴ and R⁵ are in each case hydrogens. However, additionally, it is also advantageous in some cases if in addition to the hydroxyl and the diazo group, there is at least one further substituent in the compound according to formula (I), which is not hydrogen.

Another essential feature of the compound according to formula (I) is the anion X, which is preferably tetrafluoroborate.

In connection with the group Y, the following should be noted: the group R, which is assigned to Y, is not subject to any relevant restriction. It can be established by a person skilled in the art without any problem. Especially it can be a group that corresponds to the groups R¹ to R⁸ designated above. Preferably it is an alkyl group, especially with 1 to 6 carbon atoms. In connection with the formula B(OR)₂, the group R can also be hydrogen. When Y represents Mg (halogen), magnesium chloride and bromide are preferred.

The following examples show that the compounds that are covered by the above formula (I) solve the aforementioned problem to a surprisingly favorable extent. This applies both to the yield and to the simple procedure and to the aforementioned aspect of regioselectivity.

As already mentioned, in the method according to the invention the starting compound used can be a benzenoid amide, which apart from the diazo function has the same substituents R¹, R², R³, R⁴ and R⁵. The diazo function is replaced by an amide structure —NHCO—R. With respect to the group R, the invention is not subject to any relevant restriction. R can be determined readily by a person skilled in the art. It can, for example, be a group that matches the aforementioned groups R¹ to R⁸. Preferably it is an alkyl group, especially with 1 to 6 carbon atoms. Especially the group R can also represent hydrogen. The amide is first converted by hydrolytic cleavage into an amine. Of course, the amine can alternatively also be used directly for further reaction. For this, either the amine or the amide after hydrolytic cleavage is diazotized with an organic or inorganic nitrite salt.

In order to obtain especially advantageous compounds of formula (I), the diazotized intermediate is transformed by addition of a complex anion salt with an anion in the form of BF₄⁻, PF₆⁻ and/or ClO₄⁻ into the diazonium salt, if during the diazotization an anion is used that does not correspond to these complex anions. As an example we may mention diazotization with a hydrohalic acid, for example hydrochloric acid.

Especially advantageously, the aforementioned hydrolytic cleavage is carried out with a mineral acid or fluoroboric acid. It is especially advantageous if the hydrolytic cleavage is carried out in an alcoholic solvent that contains a mineral acid, especially hydrochloric acid, or fluoroboric acid, the alcohol selected preferably being a C₁-C₄ alcohol. The alcoholic solvent is preferably methanol, ethanol, n-propanol and/or isopropanol. If an alcoholic solvent is used for the hydrolytic cleavage, it is desirable to employ an organic nitrite for the reaction, as this has greater solubility in alcoholic solvents than an inorganic nitrite. There are especial advantages in using an organic nitrite, preferably in the form of an alkyl nitrite, especially a C₁₋₆ alkyl nitrite. An alkyl nitrite with a branched alkyl group is especially advantageous. In the context of the invention, t-butylnitrite has preference.

When choosing the temperature of hydrolytic cleavage, a person skilled in the art is not especially restricted. However, it is preferable if the hydrolytic cleavage of the aforementioned amide is carried out at a temperature from about 20 to 110° C., especially from about 50 to 80° C. Similarly, there is no substantial restriction on the temperature to be selected for the diazotization. Desirably, it is from about −10 to +10° C., especially from about −5 to +5° C. It is especially advantageous if the diazotization is carried out in the reaction mixture in which hydrolytic cleavage has already been performed, i.e. there is no interposed isolation of the amine.

The especial advantage of the benzenoid diazonium salt obtained according to the invention, as presented above, is in the use as substrate in more or less any cross-coupling reactions, the advantages already mentioned above being achieved to a surprisingly favorable extent. In an especially preferred embodiment of the present invention, the term “cross coupling”, as used here, means a reaction in which, formally with the aid of a catalytically active metal complex, preferably a transition metal complex, two hydrocarbon derivatives are joined together via a new carbon-carbon bond. Heck; Suzuki, Stille, Negishi, Hiyama or Kumada coupling may be mentioned as nonlimiting examples of cross-coupling reactions. It should be mentioned that in general usage, Heck coupling is not covered by the term “cross coupling”, but should be understood as such in connection with the use described here.

As described above, in the method according to the invention at least the steps (b) and (c) should preferably be carried out without interposed isolation of an intermediate. For this, the coupling partners and the catalyst can be added to the reaction mixture before carrying out step c). Alternatively, however, it is also possible to add the coupling partner and/or the catalyst even before carrying out step b) and/or step a). It has also been found that isolation of the amine obtained after step (a) is not necessary, as the overall yield of the coupling product is not increased by isolation of the intermediate. Therefore it is advantageous to carry out all three steps (a) to (c) without interposed isolation of an intermediate.

When the term “catalyst” is used in connection with the method according to the invention, this means, of course, that it is used in small amounts, i.e. as a rule up to 5 mol. %, relative to the diazonium salt used. Especially advantageously, transition metal catalyst is used here, especially a palladium catalyst. It is especially advantageous to use Pd(OAc)₂ or Pd₂(dba)₃CHCl₃ as catalyst.

The method described above allows advantageous embodiments: Thus, it is desirable and advantageous, before carrying out the aforementioned step (b), which relates to reaction of the phenolic diazonium salt with a coupling partner and formation of the cross-coupled product, to add a base to the reaction mixture, especially a base that has good solubility in the reaction mixture, to buffer the acid that forms in step (b). The reaction mixture can advantageously be such that it contains a solvent that is based on methanol, ethanol, acetonitrile and/or water. The reaction during the cross coupling is also not substantially restricted with respect to the temperature selected. However, it is advantageous if the reaction is carried out at a temperature from about −10 to 60° C., especially from about 20 to 30° C.

The invention, presented in detail above, has many advantages over the prior art described at the beginning: In the invention, protic groups can be incorporated directly in the diazonium salt and not only in the coupling partner. The necessary amounts of metal compounds can be reduced to a catalytic amount, i.e. as a rule 5 mol. % or less, which offers a clear economic and ecological advantage. The yields and selectivities achieved according to the invention in the cross couplings described are considerably better, as the Pd-catalyzed cross couplings employed take place by other mechanisms or do not employ free radicals.

The invention is also attractive because free phenols very often occur as structural element in active substances and natural substances, so that the expenditure on protective groups during synthesis can be reduced considerably according to the invention. Thus, in nature there are numerous derivatives of E-configured hydroxystilbenes, for example resveratrol, pinosylvin and astringenin. These substances have in common that they have a strong disinfecting action. According to the invention, these useful natural substances can be produced simply and at high yield.

The invention is explained in more detail below on the basis of examples, without seeing a restriction therein.

### EXAMPLE 1

### Preparation of 4-hydroxyphenyldiazonium tetrafluoroborate

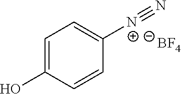

A suspension of 4-acetamidophenol (5.0 g, 33 mmol) in 3.6 N HBF₄ (15 ml) and isopropanol (5 ml) is heated for three hours at 90° C. until a clear solution is formed. The resultant solution is cooled to 0° C. and NaNO₂ (0.31 g, 4.4 mmol) is slowly added in portions. The resultant suspension is stirred for 30 min at 0° C. The solid is filtered off and the filtration residue is washed with cold diethyl ether (100 ml), giving 4-hydroxyphenyldiazonium tetrafluoroborate (4.93 g, 24 mmol) at 72% yield.

¹H NMR (300 MHz, DMSO-d₆): δ=8.31 (d, 2H, J=9.5 Hz, Ar), 6.73 (d, 2H, J=9.5 Hz, Ar).

¹³C NMR (75 MHz, DMSO-d₆, APT): δ=174.7, 134.4, 121.5, 88.6.

IR (cm⁻¹): 3098 (w), 2189 (s, N₂), 1590 (s).

MS (ESI) m/z=99 (100%), 121 (M⁺, 73%).

### EXAMPLE 2

### Preparation of 4-hydroxy-3-nitrophenyl-diazonium tetrafluoroborate

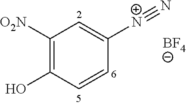

4-Hydroxy-3-nitroacetanilide (2.78 mmol, 500 mg) is put in a 50-ml one-necked flask and hydrochloric acid (5 ml, 18%) and ethanol (1 ml) are added. Then the suspension is heated for six hours under reflux, during which the solid dissolves. The solution is cooled to 0° C., the hydrochloride being precipitated. With addition of NaNO₂ (2.78 mmol, 192 mg) over a period of 15 minutes, the hydrochloride dissolves, and after stirring at 0° C. for ten minutes, NH₄BF₄ (2.78 mmol, 291 mg) is added. After 15 minutes at the latest, a solid is precipitated, which is removed with suction and is washed with cold water, ethanol and MTBE (20 ml of each). 4-Hydroxy-3-nitrophenyldiazonium tetrafluoroborate is obtained as a yellow solid at a yield of 57% (1.59 mmol, 400 mg).

¹H NMR (300 MHz, DMSO) δ=8.90 (d, J=2.9, 1H, 2-H), 7.83 (dd, J=2.9, 9.8, 1H, 6-H), 6.48 (d, J=9.8, 1H, 5-H). ¹³C NMR (75 MHz, DMSO) δ=170.3 (C-4), 140.5 (C-3), 133.6 (Ar), 131.3 (Ar), 128.3 (C-1), 79.5 (Ar). IR (cm⁻¹): 3081 (m), 2166 (s, N₂), 1597 (s), 1326 (s), 1124 (s).

MS (EI) m/z=63 (100%), 91 (35%), 139 (M⁺, 47%).

### EXAMPLE 3

### Preparation of 3-bromo-4-hydroxyphenyl-diazonium tetrafluoroborate

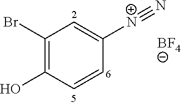

3-Bromo-4-hydroxyacetanilide (2.18 mmol, 500 mg) is put in a 50-ml one-necked flask and hydrochloric acid (4 ml, 18%) and ethanol (0.5 ml) are added. Then the suspension is heated for six hours under reflux, during which the solid first dissolves and a little later the hydrochloride is precipitated. The solution is cooled to −10° C. With addition of NaNO₂ (2.18 mmol, 150 mg) over a period of 15 minutes, the hydrochloride dissolves, and after stirring for ten minutes at −10° C., NH₄BF₄ (2.18 mmol, 229 mg) is added. After 15 minutes at the latest, a solid is precipitated, which is removed with suction and is washed with cold ethanol (5 ml) and MTBE (50 ml). 3-Bromo-4-hydroxyphenyldiazonium tetrafluoroborate is obtained as a colorless solid at a yield of 88% (1.92 mmol, 550 mg).

¹H NMR (300 MHz, DMSO) δ=8.85 (d, J=2.6, 1H, 2-H), 8.42 (dd, J=2.6, 9.3, 1H, 6-H), 7.34 (d, J=9.3, 1H, 5-H). ¹³C NMR (75 MHz, DMSO) δ=168.4 (C-4), 136.9 (Ar), 134.6 (Ar), 118.9 (Ar), 112.3 (C-3).

IR: 3145 (m), 2235 (s, N₂), 1552 (s), 1424 (s), 1101 (s).

MS (EI) m/z=63 (100%), 142 (30%), 172 (M % 44%).

### EXAMPLE 4

### Heck Reactions with Electron-Rich Aryldia-Zonium Salts

Heck reactions of the aryldiazonium salts a-c shown in Table 1 with methyl acrylate were investigated systematically.

The concrete test protocol for carrying out the tests, the results of which are given in the following Table 1, will be described first:

The corresponding diazonium salt (0.5 mmol) is added to a solution of methyl acrylate (1.0 mmol, 86 mg, 0.09 ml), NaOAc (1.5 mmol, 123 mg) and catalyst (2.5 mol. %, 3 mg) in absolute solvent (5 ml) and is stirred for 12 h at room temperature. The reaction solution was concentrated in a rotary evaporator, taken up in methyl-tert-butyl ether (MTBE) (10 ml) and washed with 1 N HCl (10 ml). The aqueous phase was extracted with MTBE (30 ml). The combined organic phases were dried over MgSO₄. The solvent was removed and the raw product was purified chromatographically.

In the tests described, two pre-catalysts (palladium acetate and the Pd₂(dba)₃.CHCl₃ complex) and two different solvents (methanol as polar-protic solvent and acetonitrile as polar-aprotic solvent) were tested. Moreover, the reaction was carried out in each case in the presence or absence of a base.

TABLE 1

| Pre-catalyst | Pd(OAc)₂ | | | | Pd₂(dba)₃CHCl₃ | | | |
|---|---|---|---|---|---|---|---|---|
| Solvent | MeOH | | CH₃CN | | MeOH | | CH₃CN | |
| Base | NaOAc | — | NaOAc | — | NaOAc | — | NaOAc | — |
| 1a (R = Bn) | 32 | 52 | 15 | 7 | 22 | 45 | 45 | 22 |
| 1b (R = Me) | 72 | 83 | 52 | 10 | 71 | 94 | 73 | 15 |
| 1c (R = H) | 99 | 95 | 99 | 40 | 99 | 95 | 99 | 21 |

Note:
The numerical values in columns 2 ff. refer to the yields achieved in each case.

The following conclusions can be drawn from the above table: The yields achieved for the benzyloxy-substituted derivative 1a are at best mediocre. As a rule, under standardized conditions, better yields are achieved in methanol than in acetonitrile. The base has a considerable influence, which, however, is opposite, depending on the solvent used: in methanol, the reactions are more successful when no base is present, but in acetonitrile much better yields are achieved in the presence of bases. It is surprising that in all test series, the best results are achieved for the unprotected diaryldiazonium salt 1c. In this case, independently of catalyst and base, quantitative yields are obtained in methanol. In acetonitrile, quantitative yields are only obtained when the base is present.

### EXAMPLE 5

As can be seen from FIG. 1 and Table 2, the three diazonium salts 1a to c were reacted under standardized conditions with three different styrenes 4a to 4c.

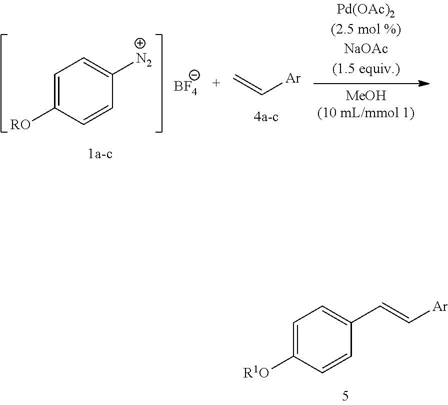

The corresponding tests are carried out according to the following protocol: the corresponding diazonium salt 1 (1.0 mmol) is added to a solution of the styrene 4 (0.5 mmol), NaOAc (1.5 mmol, 123 mg) and Pd(OAc)₂ (2.5 mol. %, 3 mg) in absolute methanol (5 ml) and stirred for 12 h at room temperature. After adding silica gel, the reaction solution is concentrated in a rotary evaporator. The raw product is purified chromatographically.

The results of the tests are shown in the following Table 2.

TABLE 2

| Styrene → Aryldiazonium salt ↓ | 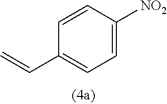 | 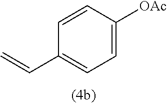 | 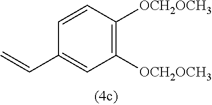 |
|---|---|---|---|
| 1a (R = Bn) | 5aa (20) | 5ab (18) | — |
| 1b (R = Me) | 5ba (79) | 5bb (38) | 5bc (80) |
| 1c (R = H) | 5ca (98) | 5cb (95) | 5cc (76) |

Note:
The percentage yields are shown in parentheses.

It can be seen that the aryldiazonium salt 1a only gives poor yields, whereas with the unprotected aryldiazonium salt, excellent yields can be achieved without exception.

### EXAMPLE 6

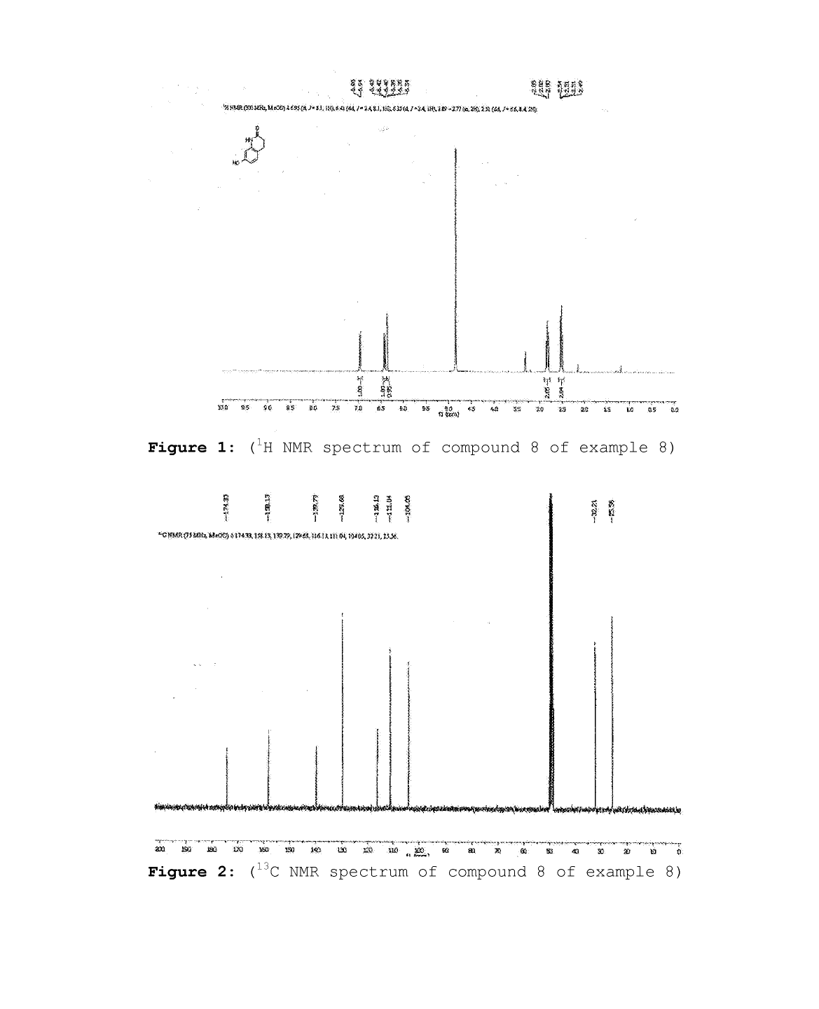

The Heck coupling of allyl alcohol (6) with the diazonium tetrafluoroborates 1b and 1c, as shown below in FIG. 2, was investigated.

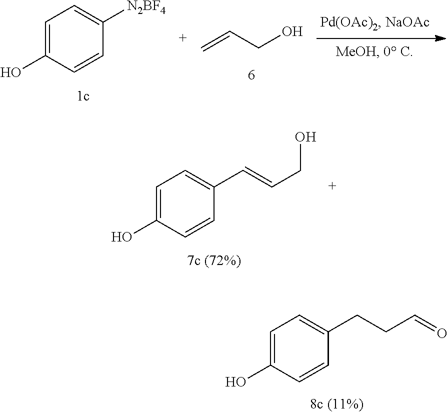

In detail, the underlying reaction is carried out as follows: The corresponding diazonium salt (1.2 mmol) is added to a solution of allyl alcohol (2.4 mmol, 0.2 ml), NaOAc (3.6 mmol, 195 mg) and Pd(OAc)₂ (5 mol. %, 14 mg) in absolute methanol (5 ml), and is stirred for 6 h at 0° C. The reaction solution is concentrated in a rotary evaporator, taken up in MTBE (10 ml) and washed with 1 N HCl (10 ml). The aqueous phase is extracted with MTBE (30 ml). The combined organic phases are dried over MgSO₄. The solvent is removed and the raw product is purified chromatographically (solvent: cHex:MTBE 1:1).

p-Cumaryl alcohol 7c was obtained at 72% yield. The aldehyde 8c resulting from an isomerizing Heck coupling was formed as by-product at 11% yield.

While carrying out the above tests, another interesting finding was made, as described below:

By changing the solvent to acetonitrile and adding a strong donor (chloride, in the form of tetrabutylammonium chloride), the selectivity of the reaction can be reversed. Under these conditions, only 8c is obtained, at 69% yield. Increasing the isomerization activity by adding halides in Heck couplings has already been described previously, but not for diazonium salts as coupling partner (“Tetrahedron Lett.” 1989, 30, 2603-2606). When using the methoxy derivative 1b, the alcohol 7b, analogous to 7c, is obtained in methanol. However, in acetonitrile in the presence of tetrabutylammonium chloride, when using 1b, no product can be isolated, so that also in this example, an altogether higher activity of the unprotected para-hydroxyphenyldiazonium salt 1c can be assumed.

### EXAMPLE 7

A Suzuki coupling of the phenyltrifluoroborate 1c was reacted, according to Drawing 3, with potassium phenyltrifluoroborate in methanol using Pd(OAc)₂ as catalyst to (1,1′-biphenyl)-4-ol. The alcohol was obtained at 51% yield.

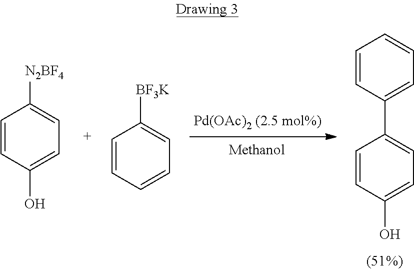

In this reaction, in contrast to the method described in “Tetrahedron Lett.”, 1979, 657-660, there is no regioselectivity problem. Moreover, only a catalytic amount of metal salt is used and the yield is far higher.

### EXAMPLE 8

### One-Pot Deacetylation-Diazotization Heck Reaction; Heterocyclic Nucleus of Aripiprazole

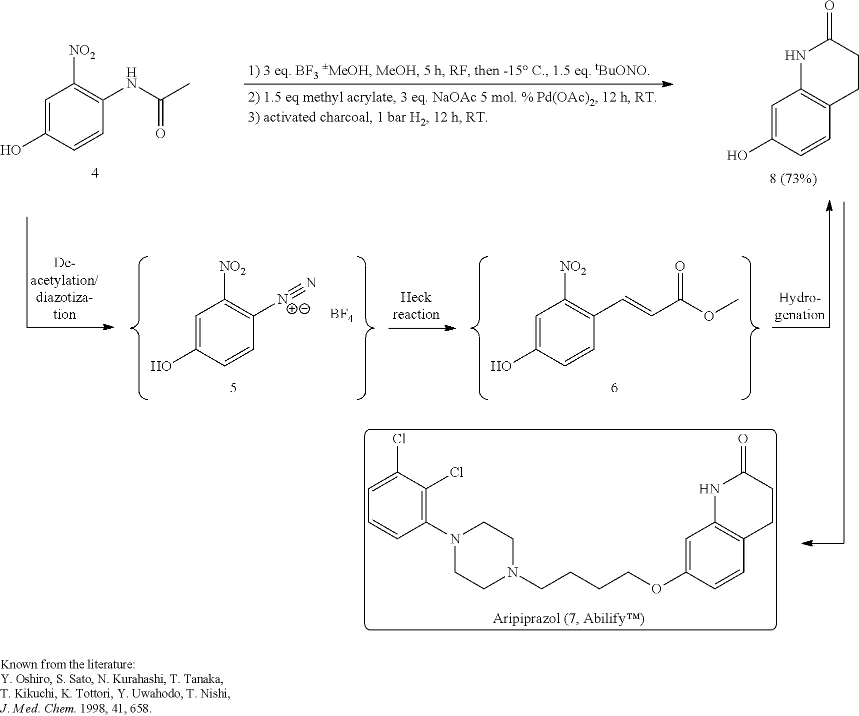

Preparation of 7-hydroxy-3,4-dihydroquinolin-2(1H)-one (8): 4-Hydroxy-2-nitroacetanilide (4) (3.03 mmol, 594 mg) is put in a 25-ml two-necked flask under nitrogen atmosphere and absolute MeOH (5 ml) is added. Then BF₃.MeOH (9.08 mmol, 0.98 ml, 50/50) is added and the solution is heated for five hours under reflux. The solution is cooled to −15° C. After addition of tert-butyl nitrite (4.54 mmol, 0.54 ml) it is stirred for 20 minutes, slowly raising the temperature to 0° C.

After addition of sodium acetate (9.08 mmol, 745 mg), a solid precipitates, which is dissolved by adding methanol (5 ml).

Then methyl acrylate (4.54 mmol, 391 mg, 0.41 ml) and palladium acetate. (5 mol. %, 0.15 mmol, 34 mg) are added. The solution is stirred for 12 hours at room temperature. Activated charcoal (85 mg) is added to the solution and the reaction mixture is stirred for 24 hours under hydrogen atmosphere (1 bar). The reaction mixture is filtered on Celite, HCl solution (1N) is added to the filtrate and it is shaken three times with ethyl acetate (20 ml each time).

The combined organic extracts are dried over magnesium sulfate, the drying agent is filtered off and the solvent is removed under vacuum. After purification by column chromatography (MTBE), 7-hydroxy-3,4-dihydroquinolin-2(1H)-one (8) is obtained as a colorless solid at a yield of 73% (2.21 mmol, 360 mg).

¹H NMR (300 MHz, MeOD) δ 6.95 (d, J=8.1, 1H), 6.41 (dd, J=2.4, 8.1, 1H), 6.35 (d, J=2.4, 1H), 2.89-2.77 (m, 2H), 2.51 (dd, J=6.6, 8.4, 2H). ¹³C NMR (75 MHz, MeOD) δ 174.3, 158.1, 139.8, 129.7, 116.1, 111.0, 104.0, 32.2, 25.6. MS (ESI) m/z=99 (13%), 122 (5%), 164 (100%). HRMS (ESI) m/z for C₉H₁₀NO₂ [M+H]⁺; calculated 164.0712. found 164.0721. Elemental analysis for C₉H₉NO₂: calculated C, 66.25%; H, 5.56%; N, 8.58% found C, 65.98%; H, 5.60%; N, 8.40%. M.p. 233° C. FIG. 1 given below shows a ¹H NMR spectrum and FIG. 2 shows a ¹³C NMR spectrum of the above compound 8.

### EXAMPLE 9

### Example of Base-Free Heck Reaction. (E)-Methyl 2-hydroxy-5-(3-methoxy-3-oxoprop-1-enyl)benzoate (2)

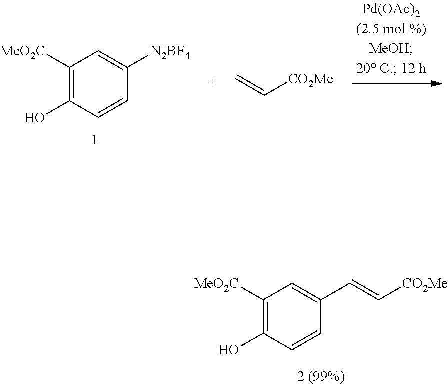

Diazonium salt 1 (266 mg, 1.0 mmol) is dissolved in dry methanol (5.0 ml) under a nitrogen atmosphere. Methyl acrylate (170 mg, 2.0 mmol) is added, followed by Pd(OAc)₂ (5.6 mg, 2.5 mol. %). The reaction mixture is stirred at 20° C. for 12 hours. Then activated charcoal (50 mg) is added, to bind the catalyst residues. All volatile components are removed under vacuum, and the residue is extracted with ethyl acetate (25 ml). After filtration over Celite, the solvent is removed and the residue is purified by silica gel chromatography (eluent hexane/ethyl acetate). Yield: 234 mg (99%). Colorless solid, m.p. 94-96° C.

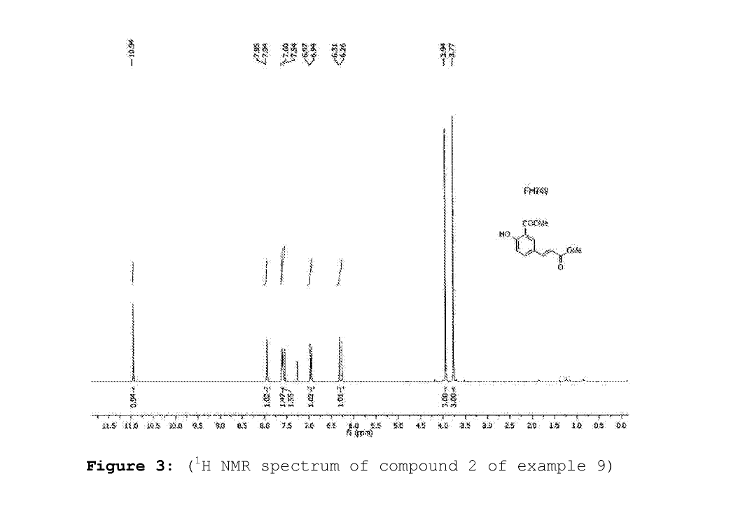

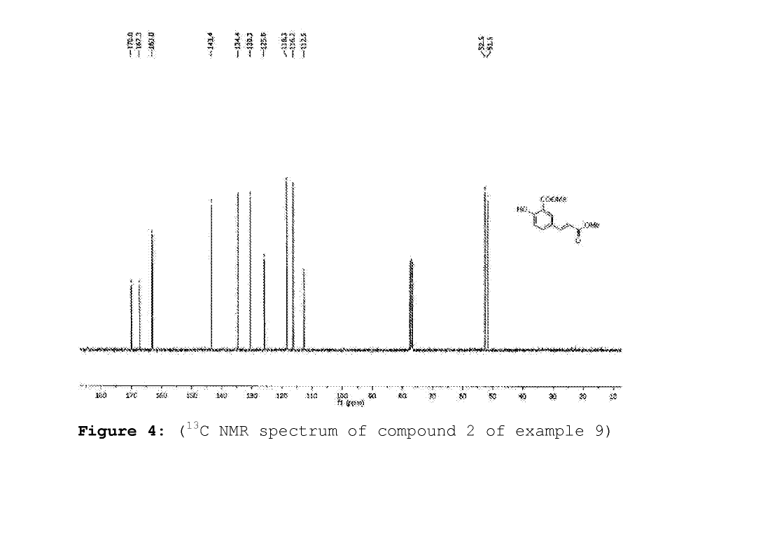

¹H NMR (300 MHz, CDCl₃) δ 10.94 (s, 1H), 7.94 (d, J=2.0, 1H), 7.59 (dd, J=8.6, 2.2, 1H), 7.57 (d, J=16.0, 1H), 6.95 (d, J=8.7, 1H), 6.29 (d, J=16.0, 1H), 3.94 (s, 3H), 3.77 (s, 3H); ¹³C NMR (75 MHz, CDCl₃) δ 170.0 (O), 167.3 (O), 163.0 (O), 143.4 (1), 134.4 (1), 130.3 (1), 125.8 (O), 118.3 (1), 116.2 (1), 112.5 (O), 52.5 (3), 51.5 (3); IR (neat, cm⁻¹): v 3155 (w), 2953 (w), 1712 (m), 1675 (s), 1635 (s), 1608 (m), 1592 (m), 1491 (m), 1440 (s), 1352 (m), 1308 (m), 1287 (m), 1203 (s), 1167 (s); MS (ESI): m/z 227 ([M+H]⁺, 18); 205 (100); HRMS (ESI): calcd for C₁₂H₁₃O₅ [M+H]⁺: 237.0763. found: 237.0766; Anal. calcd for Cl₁₂H₁₂O₅: C, 61.0; H, 5.1. found: C, 60.9; H, 4.9. The following FIGS. 3 and 4 show the ¹H NMR and ¹³C NMR spectra of the above compound 2.

1. A method for producing a cross-coupling product using a benzenoid diazonium salt according to general formula (I) 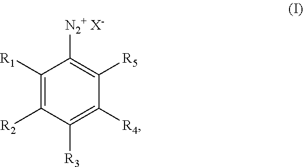
where the groups R₁, R₂, R₃, R₄ and R₅, independently of one another, represent hydrogen, halogen, an alkyl, alkenyl, aryl, alkoxy, aryloxy, nitro, cyano, hydroxyl, acetyl and/or diazo group and X represents BF₄, Cl, F, SO₃CH₃, CO₂CH₃, PF₆, ClO₂CH₃ or ClO₄, comprising the steps
(a) providing a benzenoid amide, which with the exception of the diazo function has the same substituents R₁, R₂, R₃, R₄ and R₅ as the benzenoid diazonium salt of general formula (I), and hydrolytically cleaving the amide to an amine or providing a corresponding amine,
(b) diazotizing the amine thus obtained or provided with a nitrite and
(c) then reacting the benzenoid diazonium salt with a coupling partner in the presence of a catalyst with formation of a cross-coupling product, the coupling partner being represented by general formula (II) 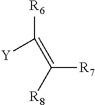
R₆, R₇ and R₈ are identical or different and represent hydrogen, carboxyalkyl groups, carboxyaryl groups, alkyl groups, aryl groups, alkoxy groups, aryloxy groups, wherein the groups can in each case contain Si, N, S, O and/or halogen atoms, or R₆ and R₇ form, with the double bond, an aromatic ring, which can be provided with R₈ and one to four further substituents, independently of one another, selected from the group comprising a linear or branched (C₁-C₆)-alkyl group, a (C₃-C₇)-cycloalkyl group, a linear or branched (C₁-C₆)-alkenyl group, a linear or branched (C₁-C₆)-alkoxy group, halogen, the hydroxyl group, an amino, di(C₁-C₆)-alkylamino, nitro, acetyl, cyano, benzyl, 4-methoxybenzyl, 4-nitrobenzyl, phenyl and 4-methoxyphenyl group, and Y=H, —B(OR)₂, —SnR₃, —ZnR, —SiR₃ or Mg (halogen) and wherein at least the steps (b) and (c) are carried out without interposed isolation of an intermediate.

2. A method according to claim 1, characterized in that all three steps (a) to (c) are carried out without interposed isolation of an intermediate.

3. A method according to claim 1, characterized in that the benzenoid diazonium salt is a phenolic diazonium salt.

4. A method according to claim 1, characterized in that the hydroxyl group of the phenolic diazonium salt does not have a protective group.

5. A method according to claim 1 characterized in that the diazotized intermediate obtained in step (b) is transformed by addition of a complex anion salt with an anion in the form of BF₄⁻, PF₆⁻ and/or ClO₄⁻ into the diazonium salt.

6. A method according to claim 1, characterized in that the hydrolytic cleavage in step (a) is carried out with a mineral acid or fluoroboric acid.

7. A method according to claim 1, characterized in that the hydrolytic cleavage in step (a) is carried out in an alcoholic solvent that contains a mineral acid, especially hydrochloric acid or fluoroboric acid, the alcohol being especially a C₁-C₄ alcohol.

8. A method according to claim 7, characterized in that methanol, ethanol, n-propanol and/or isopropanol are used as alcoholic solvent.

9. A method according to claim 1, characterized in that the hydrolytic cleavage of the amide in step (a) is carried out at a temperature from about 20 to 110° C., especially from about 50 to 80° C.

10. A method according to claim 1, characterized in that the diazotization in step (b) is carried out from about −10 to +10° C., especially from about −5 to +5° C.

11. A method according to claim 1, characterized in that a transition metal catalyst, especially a palladium catalyst, is used as catalyst in step (c).

12. A method according to claim 11, characterized in that Pd(OAc)₂ or Pd₂(dba)₃CHCl₃ is used as catalyst.

13. A method according to claim 1, characterized in that before carrying out step (b), a base, especially a base that dissolves well in the reaction mixture, is added to the reaction mixture for buffering the acid that forms in step (b).

14. A method according to claim 1, characterized in that the reaction of step (c) is carried out in a solvent that is based on methanol, ethanol, acetonitrile and/or water.

15. A method according to claim 1, characterized in that the reaction of step (c) is carried out at a temperature from about −10 to 60° C., especially from about 20 to 30° C.
